(19) United States
(12) Patent Application Publication
(10) Pub. No.: US 2026/0192217 A1
(43) Pub. Date: Jul. 9, 2026

(54) SCALABLE FLUIDIC DEVICES FOR HIGH-THROUGHPUT ENRICHMENT AND SEPARATION OF CHARGED SPECIES AND METHODS OF USE

(71) Applicant: Iowa State University Research Foundation, Inc., Ames, IA (US)

(72) Inventors: Robbyn K. Anand, Ames, IA (US); Beatrise Berzina, Ames, IA (US); Zisun Ahmed, Ames, IA (US); Umesha Peramune, Ames, IA (US)

(21) Appl. No.: 19/439,874

(22) Filed: Jan. 5, 2026

Related U.S. Application Data
(60) Provisional application No. 63/741,553, filed on Jan. 3, 2025.

(51) Int. Cl. B01D 15/38; A61M 1/14; B01D 15/36; C02F 1/42; C02F 1/48; C02F 103/08

(52) U.S. Cl. CPC B01D 15/3885; A61M 1/14; B01D 15/362; C02F 1/42; C02F 1/48; C02F 2001/425; C02F 2103/08

(57) ABSTRACT

The present disclosure relates generally to fluidic devices for high-throughput removal of charged particles from a fluid, for example contaminants from drinking water or contaminants from spent dialysate. The fluidic devices comprise a flow-through 3D ion selective structure within a channel for focusing charged particles along an electric field gradient at the boundary of an ion depleted zone and/or in an ion enriched zone created by the 3D ion selective structure, thereby removing said charged particles from the fluid flowing through the 3D ion selective structure.

### CROSS-REFERENCE TO RELATED APPLICATIONS

This application claims priority under 35 U.S.C. § 119 to provisional application Ser. No. 63/741,553, filed Jan. 3, 2025, herein incorporated by reference in its entirety.

### STATEMENT REGARDING FEDERALLY SPONSORED RESEARCH OR DEVELOPMENT

This invention was made with government support under National Science Foundation Grant No. 2344398. The government has certain rights in the invention.

### TECHNICAL FIELD

The present disclosure relates generally to a device for high-throughput removal of charged and neutral particles from a fluid, for example contaminants from drinking water or contaminants from fluid employed for hemodialysis or peritoneal dialysis (“spent dialysate”). The present disclosure also relates generally to a device for enrichment of chemical species, such as rare earth metals, from groundwater or water bodies. The device utilizes a 3D flow-through ion selective structure for continuous separation of charged species and separates said charged species into a physically separated stream, thereby removing said species from a flowing fluid.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Ion concentration polarization (ICP) is an electrokinetic phenomenon occurring when an electrical bias is applied across an ion-selective structure. An ion selective feature can be planar or 3-dimensional, e.g., electrodes, membranes, honeycomb, mesh, bead beds, discs, or any combination thereof. When a potential is applied across an ion selective feature, ion depletion zones (IDZ) and ion enrichment zones (IEZ) are created. The low ionic conductivity of the IDZ leads to a strong (>10 fold) local enhancement of the electric field and the formation of the electric field gradient. The non-linear migration of ions in these gradients results in further exclusion of charged species from entering the IDZ, as shown in FIG. 2. The steep electric field gradient at the IDZ boundary has been employed for concentration enrichment and separation of charged species.

Previous planar and membrane-based electrokinetic water desalination and purification devices using ICP have insufficient throughput for practical applications due to the instability of generated electric field and electro-convective flow, specifically vortices forming near the ion selective features.

In microfluidic applications, 3D beds of charged microbeads are currently utilized in electrokinetic focusing of charged species for biosensing applications. These systems use a set of flow-through packed beds of microbeads to achieve perm-selective ion transport and geometrically restrict the formation of large vortices, leading to the formation of stabilized IDZ boundary, in microfluidic devices. However, scalability remains a challenge. Furthermore, these devices enrich into a focused plug and sense analytes at an electric field gradient. There remains a need for continuous separation of charged species into a physically separated stream, thereby removing said species from a flowing fluid.

Accordingly, it is an objective of the disclosure to provide electrokinetic devices, and methods of using said devices, that employ ICP to separate charged species from a fluid in a flow-through device that utilizes a 3D ion selective structure. Such devices and methods can be used, for example, for water purification or dialysate regeneration.

It is a further objective of the disclosure to provide portable modular electrokinetic water purification devices that employ ICP to separate charged species from a fluid in a flow-through device that utilizes a 3D ion selective structure.

It is still a further objective of the disclosure for said devices to optionally comprise in-line sensing for said charged species and/or water contaminants.

Other objects, advantages and features of the present invention will become apparent from the following specification taken in conjunction with the accompanying examples or drawings.

### BRIEF SUMMARY

Disclosed herein are fluidic devices comprising a main channel connected to at least one inlet and at least one outlet, wherein the main channel is configured to flow a liquid through the main channel from inlet to outlet, wherein the inlet and/or the outlet is connected to an electrode; at least one flow-through 3D ion selective structure within the main channel comprising microbeads, wherein the microbeads are contained within a bead bed which extends at least a portion of the width and length of the main channel as defined by a bead bed structure; and one or more of the following: a) a side channel configured to flow a portion of the liquid within the main channel from the main channel to a side outlet, wherein the side channel is located upstream from the 3D ion selective structure; and b) a secondary channel adjacent to the main channel and in fluid connection with the main channel, wherein at least a portion of the bead bed and bead bed structure extends into at least a portion of the secondary channel.

In some embodiments, the main channel further comprises at least one secondary bead bed comprising microbeads, wherein the microbeads are contained within the secondary bead bed defined by a bead bed structure within the main channel, wherein the secondary bead bed is located upstream from the flow-through 3D ion selective structure.

In some embodiments, the device further comprises a power source connected with the inlet electrode and/or the outlet electrode and/or the 3D ion selective structure, wherein the power source is configured to supply a voltage in the range of from about 1 V to about 1000 V.

Also disclosed herein are methods of removing charged species from a liquid comprising
flowing a liquid containing at least one charged species through the main channel of the fluidic device described herein; applying a voltage to the inlet and/or outlet and/or the 3D ion selective structure for a period of time focusing the charged species along an electric field gradient at the boundary of an ion depleted zone and/or in an ion enriched zone created by the 3D ion selective structure; and removing at least a portion of the focused charged species from the liquid, into the side channel and/or into the secondary channel.

In some embodiments, the liquid is fresh water, seawater, blood, blood plasma, or dialysate.

Also disclosed herein are water purification systems or a kidney dialysis systems comprising the fluidic devices described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows an illustration of a branched fluidic device with an in-line 3D ion selective structure comprised of SiC discs and a bead bed of sulfonated divinylbenzene (DVB) beads.

FIG. 2A is a schematic drawing showing the ICP phenomenon, and the formation of ion depletion and ion enrichment zones in two parallel fluidic channels interconnected with an ion selective structure after application of an electric bias.

FIG. 2B is a schematic drawing illustrating the ICP phenomenon, and the formation of ion depletion and ion enrichment zones, in split fluidic channels connected with an ion selective structure after application of an electric bias.

FIG. 3 is a schematic showing the formation of an IDZ and IEZ and purification of water downstream.

FIG. 4 is a schematic of a top view of a 3D printed packed bed fluidic water purifier.

FIGS. 5 & 6 are fluorescence micrographs demonstrating electrokinetic focusing of an anionic dye (BODIPY²⁻) in a scaled-up electrokinetic preconcentration device.

FIG. 5 is the fluorescence micrograph at time 0, before a voltage is applied.

FIG. 6 is the fluorescence micrograph 10 minutes after the voltage is applied.

FIG. 7 is a graph of percent dye leakage over time.

FIG. 8 is a graph of conductance over time.

FIG. 9 is a picture of a device fabricated using thermoplastics and sealed with epoxy adhesive, housing two porous SiC disks and DVB beads in a 10 mm diameter channel.

FIG. 10 is a current-voltage curve obtained by applying voltage in the inlet and outlet reservoirs across the 3D structure.

FIG. 11 is a graph of conductivity over time when continuous potential is applied for 15 minutes.

FIG. 12 is an illustration of an exemplary gravity-driven seawater desalination system.

FIG. 13 is a schematic illustration of a modular water purification system.

FIG. 14 is a schematic illustration of an electrokinetic purification module with optional sensing modules.

FIG. 15 is an illustration (left) and a photograph (right) of a gravity driven fluidic device with a branched waste channel.

FIG. 16 is an illustration of a fluidic device with a branched channel.

FIG. 17 contains fluorescence micrographs showing the location of tracers under 40V. The micrographs on the right, labeled a and b, are for undiluted blood plasma samples and the micrographs on the left, labeled c and d, are for model blood plasma containing 0.1 % of the albumin content of blood plasma.

FIG. 18 is an illustration of a fluidic device wherein the side channel is “on axis” and the main channel is diverted. FIG. 18 includes a side view on the left and two top views on the right.

FIG. 19 is an illustration of an exemplary electrode placement for the device illustrated in FIG. 18.

FIG. 20 is an illustration of a water purification device with in-line 3D ion selective structure consisting of a 3D printed mesh and SiC discs, and symmetric waste outlet ports.

FIG. 21 is a graph of conductivity over time of the output water in a water purification device.

FIG. 22 is an illustration of a water purification device with in-line 3D ion-selective structure and split main fluidic channel. This illustration shows the interior of the device (split in half) and indicates the placement of the main channel, the waste (or side) channel, a bead bed, slits for porous discs, and ports.

FIG. 23 is a CVC curve obtained using 90 μL/min withdraw flow rate.

FIG. 24 is a CVC curve obtained using 30 μL/min withdraw flow rate.

FIG. 25 is a graph of conductivity over time using a 30 μL/min withdraw flow rate.

FIG. 26 is a CVC curve obtained using 90 μL/min withdraw flow rate.

FIG. 27 is a CVC curve obtained using 30 μL/min withdraw flow rate.

FIG. 28 is a graph of conductivity over time using a 30 μL/min withdraw flow rate.

FIG. 29 is a CVC curve obtained using 30 μL/min withdraw flow rate.

FIG. 30 is a graph of conductivity over time using a 30 μL/min withdraw flow rate.

FIG. 31 is a graph of salt removal efficiency, depicted as a percent increase over time.

FIG. 32 is a CVC curve obtained using 30 μL/min withdraw flow rate.

FIG. 33 is a graph of conductivity over time using a 30 μL/min withdraw flow rate.

FIG. 34 is a graph of salt removal efficiency, depicted as a percent increase over time.

FIG. 35 is a graph of conductivity over time.

FIG. 36 is a graph of salt removal efficiency, depicted as a percent increase over time.

FIG. 37 is a CVC curve obtained using low flow or no-flow conditions.

FIG. 38 is a graph of conductivity over time at 80 V driving voltage.

FIG. 39 is a graph of conductivity over time at 100 V driving voltage.

FIG. 40 is an illustration of a water purification device with an in-line 3D ion selective structure with a lengthened bead bed, and split main fluidic channel. This illustration shows the interior of the device (split, showing two halves) and indicates the placement of the main channel, the waste (or side) channel, a bead bed, slits for porous discs, and various ports.

FIG. 41 is a CVC curve obtained using 30 μL/min withdraw flow rate.

FIG. 42 is a graph of conductivity over time at 100 V driving voltage.

FIG. 43 is a graph of salt removal efficiency, depicted as a percent increase over time.

FIG. 44 is an illustration of a water purification device with in-line 3D ion-selective structure and a symmetrically split main fluidic channel. The ion-selective structure has three porous discs and two separate bead beds. This illustration shows the interior of the device (split in half) and indicates the placement of the main channel, the waste (or side) channels, a bead bed, slits for porous discs, and ports.

FIG. 45 is a CVC curve obtained using low flow or no-flow conditions.

FIG. 46 is a graph of conductivity over time with driving voltages of 80 V and 100 V.

FIG. 47 is a graph of salt removal efficiency, depicted as a percent increase over time, with driving voltages of 80 V and 100 V.

FIG. 48 is a graph of conductivity over time with driving voltages of 80 V and 120 V.

FIG. 49 is a graph of salt removal efficiency, depicted as a percent increase over time, with driving voltages of 80 V and 120 V.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

The present disclosure relates to scalable fluidic devices, and methods of use, for high-throughput enrichment and separation of charged and/or neutral species from a liquid. The fluidic devices utilize a 3D ion selective structure within a main channel. The 3D ion selective structure comprises microbeads and is used to electrokinetically focus and separate charged species from the fluid flowing through the main channel and remove the focused charged species from the from the fluid for example into either a side channel or a secondary channel. Additionally, in some embodiments, neutral species depletion from a liquid is achieved. Beneficially, in the fluidic devices described herein, the electric field is distributed across the whole channel cross-section, flow is stabilized, and the system is scalable

The embodiments described herein are not limited to any particular device or method of using the device, which can vary and are understood by skilled artisans based on the present disclosure herein. It is further to be understood that all terminology used herein is for the purpose of describing particular embodiments only, and is not intended to be limiting in any manner or scope. For example, as used in this specification and the appended claims, the singular forms “a,” “an,” and “the” can include plural referents unless the content clearly indicates otherwise. Further, all units, prefixes, and symbols may be denoted in its SI accepted form.

Numeric ranges recited within the specification are inclusive of the numbers within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (e.g. 1 to 5 includes 1, 1.5, 2, 2.75, 3, 3.80, 4, and 5).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation. The preferred materials and methods are described herein. In describing and claiming the embodiments of the present invention, the following terminology will be used in accordance with the definitions set out below.

The term “about,” as used herein, refers to variations in size, distance or any other types of measurements that can be resulted from inherent heterogeneous nature of the measured objects and imprecise nature of the measurements itself. The term “about” also encompasses variation in the numerical quantity that can occur, for example, through typical measuring or handling procedures in the real world; through inadvertent error in these procedures; through differences in the manufacture, source, or purity of the ingredients used to make the device or carry out the methods, and the like. Whether or not modified by the term “about”, the claims include equivalents to the quantities.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

The methods and compositions of the present invention may comprise, consist essentially of, or consist of the components and ingredients of the present invention as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

### Fluidic Devices

The present disclosure relates to a fluidic device. The device comprises an inlet and an outlet, both in fluid connection with at least one channel. The channel is configured to retain and move a liquid from inlet to outlet. As used herein, “liquid” and “fluid” maintain their common definitions as known in the art and are used herein interchangeably (i.e. a liquid is a fluid that is flowable and conforms to the shape of its container). In another aspect, the present disclosure provides a fluidic device comprising at least one flow-through 3D ion selective structure within the channel.

In an embodiment, the 3D ion selective structure comprises microbeads. In an embodiment the 3D ion selective structure comprises conductive microbeads. In another embodiment, the microbeads are not conductive. In an embodiment, the 3D ion selective structure comprises charged microbeads. In an embodiment, the 3D ion selective structure comprises charged microbeads that are not conductive. In an embodiment, the microbeads are contained within the channel as defined by a bead bed structure. As used herein, a bead bed structure comprises a structure in which to define the area and location of the microbeads and to contain the beads. The structure can be anything as known in the art to define the area and location of the microbeads and to retain the beads within at least a portion of the channel while maintaining flow of the fluid through the channel. In a preferred embodiment, the bead bed structure comprises bead bed posts which extend from a side (e.g. a floor, ceiling, side, etc.) of the channel and are spaced such that the microbeads are contained within the area defined by the bead bed posts. In an embodiment, the bead bed structure comprises microslits. In another embodiment, the structure is a weir structure. In yet another embodiment, the structure is a porous matrix or material. In yet another embodiment, the microbeads are bound together by a chemical linker. In a preferred embodiment, the bead bed structure comprises porous ceramic plates. In another preferred embodiment, the bead bed structure comprises porous glass. In another preferred embodiment, the bead bed structure comprises nylon mesh.

In an embodiment, the 3D ion selective structure comprises metallic porous plates, a metal mesh, and/or another inorganic conductor/semiconductor. In these embodiments, a voltage can be applied directly to the ion selective structure. In an embodiment, the 3D ion selective structure comprises an inverse opal, a microporous plate, or a fibrous network with nano-to-microscale porosity. The 3D ion selective structure comprises surface chemistry that yields a high surface charge. In some of these embodiments, the 3D ion selective structure does not comprise microbeads.

In some embodiments, the 3D ion selective structure is configured for micellar electrokinetic capillary chromatography (MEKC) wherein formed micelles act as pseudo-stationary phase and interact with analytes based on their hydrophobicity. In this embodiment, neutral species are incorporated into the micelle and electrophoretically separated due to the charge of the micelles.

In an embodiment, the 3D ion selective structure comprises an electrode wherein the electrode extends across at least a portion of the channel with a portion outside of the channel for electrical connection. In an embodiment, the electrode is a planar band. In another embodiment, the electrode is a conductive rod or wire. In yet another embodiment, the electrode is a conductive epoxy or ionic liquid. In an embodiment, the electrode is a wire mesh, a porous metal plate, a packed bed of conductive beads, or an ultra-high surface area capacitor. In an embodiment, the electrode is a comb-like interdigitated or layered structure that penetrates the ion selective structure. In a preferred embodiment, the electrode comprises the ultra-high surface area capacitor and/or the comb-like interdigitated or layered structure that penetrates the ion selective structure. With these embodiments, the capacitor supplies current via capacitive charging instead of by electrochemical reactions, thereby avoiding unwanted side products. The comb-like structure improves the efficiency and stability of selective ion transport.

In an embodiment, electrodes that apply the voltage are located within the 3D ion selective structure or can be separated from the 3D ion selective structure (upstream or downstream). In a preferred embodiment, the fluidic device comprises an electrode within the 3D ion selective structure and an electrode at the outlet. This embodiment avoids contact between the electrode and charged particles in the fluid. In a preferred embodiment, the fluidic device comprises an electrode within the 3D ion selective structure and an electrode at the inlet.

In an embodiment, the channel further comprises at least one secondary bead bed comprising microbeads. In an embodiment, the secondary bead bed comprises nonconductive microbeads. In an embodiment, the secondary bead bed is placed closer to the inlet than the 3D ion selective structure (“upstream”). This at least one secondary bead bed comprises a structure defining the size of the bead bed and containing the beads. The structure can be anything as known in the art to define the bead bed area and retain the beads in the bead bed while maintaining flow of the liquid through the channel. In an embodiment, the structure comprises bead bed posts which extend from the floor of the channel and are spaced such that the microbeads are contained within the area defined by the bead bed posts. In an embodiment, the structure comprises microslits. In another embodiment, the structure is a weir structure. In yet another embodiment, the structure is a porous matrix or material. In yet another embodiment, the microbeads are bound together by a chemical linker. In an embodiment, the secondary bead bed and the 3D ion selective structure are adjacent. In an embodiment, the secondary bead bed and the 3D ion selective structure share at least a portion of the same bead bed structure.

In some embodiments, the secondary bead bed is configured for micellar electrokinetic capillary chromatography (MEKC) wherein formed micelles act as pseudo-stationary phase and interact with analytes based on their hydrophobicity. In this embodiment, neutral species are incorporated into the micelle and electrophoretically separated due to the charge of the micelles.

In an embodiment, the main channel comprises more than one 3D ion selective structure and/or more than one secondary bead bed.

In an embodiment, more than one, or several, main channels are in fluid connection to a singular inlet and a singular outlet. In an embodiment, more than one, or several, main channels are in fluid connection to a singular inlet and more than one outlet. In an embodiment, more than one, or several, main channels are in fluid connection to more than one inlet and a singular outlet. In an embodiment, more than one, or several, main channels are in fluid connection to the same inlet and/or outlet as any other main channel. Any main channel may have a unique inlet. Any main channel may have a unique outlet. Any main channel may have the same inlet or outlet as any other main channel.

The flow-through 3D ion selective structure in any one main channel may be comprised of the same materials as another 3D ion selective structure in a different main channel, or may comprise different materials. In an embodiment, each main channel may comprise one or more, or several, 3D ion selective structures and may further comprise one or more secondary bead bed. In an embodiment, the flow-through 3D ion selective structure of each channel has its own electrical connection. In an embodiment, any 3D ion selective structure may share the same electrical connection of any other 3D ion selective structure in a different main channel.

In an embodiment, a number of main channels can be grouped together and then connected fluidly with another group(s) of main channels. Within each group of main channels, any two main channels can be parallel to each other, on top of each other, or in another arrangement.

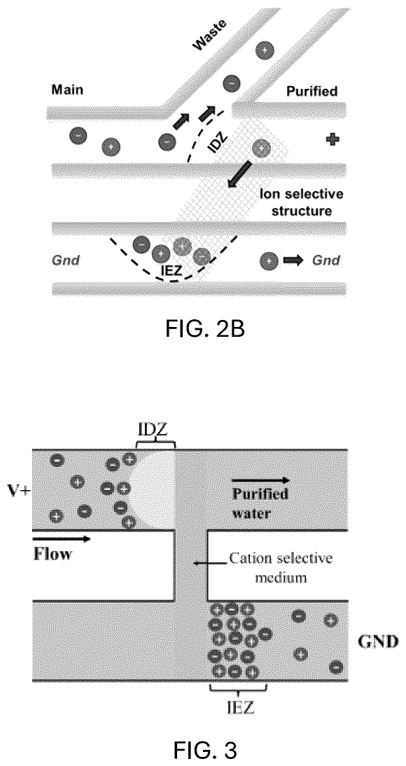

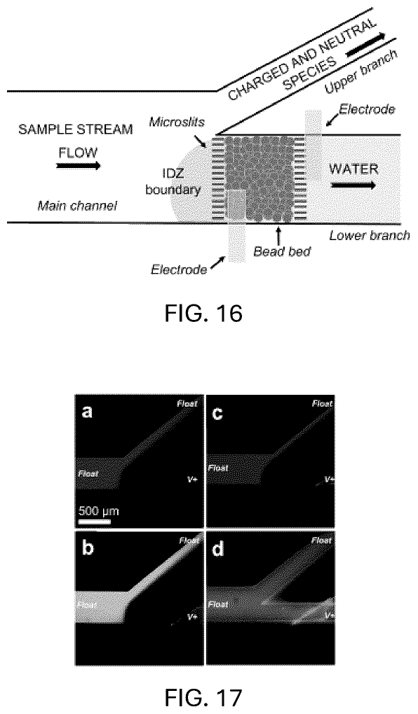

In an embodiment, the fluidic devices as described herein comprise a side channel and/or a secondary channel for removal of the charged species from the main channel and the fluid therein. In some embodiments, the secondary channel is configured to focus charged particles in an IEZ as illustrated in FIG. 2A, FIG. 2B and FIG. 3. In some embodiments, the side channel is configured to focus charged species at the boundary of an IDZ as illustrated in FIG. 2B and FIG. 16.

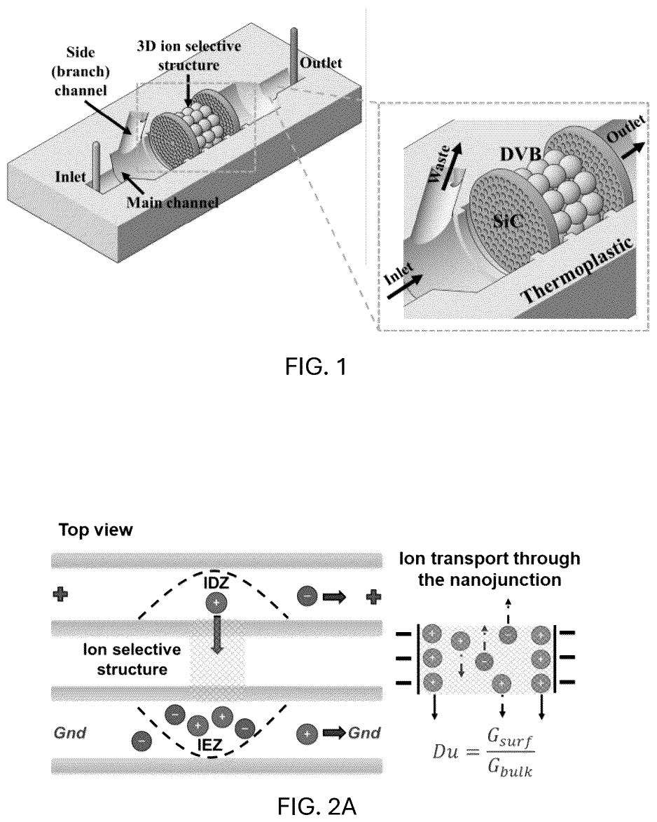

An exemplary device comprising a side channel is illustrated in FIG. 1. This figure illustrates a fluidic device comprising a main channel configured to flow a liquid from an inlet to an outlet, the inlet and outlet configured for electrical connection. Within the main channel is a 3D ion selective structure comprising conductive sulfonated divinylbenzene (DVB) microbeads and a bead bed structure comprising porous SiC plates. This exemplary fluidic device comprises a side channel configured to divert a portion of fluid within the main channel to a side outlet. The side channel is positioned upstream of the 3D ion selective structure. For this exemplary fluidic device, it is configured for a voltage bias to be applied between the inlet and outlet, across the 3D ion selective structure while a liquid flows from inlet to outlet. The voltage bias drives charge transfer reactions developing ion concentration polarization (ICP) and the formation of an ion depleted zone (“IDZ”) allowing for counter-flow focusing of charged species. The charged species will collect at the boundary of the IDZ where the side channel is positioned. The side channel will divert the liquid concentrated with the charged species away from the main channel.

Another exemplary device comprising a side channel is illustrated in FIG. 15. This figure illustrates a middle portion of a main channel comprising a 3D ion selective structure comprising charged beads and a bead bed structure comprising porous plates. A voltage is applied across the inlet and outlet, illustrated with a “+” toward the inlet (not shown) and a “−” toward the outlet (not shown). In this example, the flow of liquid through the fluidic device is gravity driven. The voltage bias drives the formation of an IDZ concentrating the charged particles at the IDZ boundary. The side channel is positioned where the charged particles concentrate and divert said charged species away from the main channel, reducing the amount of charged particles from the fluid. The exemplary device illustrated in FIG. 15 is therein being utilized for water purification. Water comprising charged species (for example, seawater or contaminated water, labeled “Feed”) flows from the inlet through the 3D ion selective structure and toward the outlet. ICP focuses the charged particles at the boundary of the IDZ and into the side “waste” channel and purified water (“Fresh”) flows through the 3D ion selective structure and toward the outlet.

Another exemplary device comprising a side channel is illustrated in FIG. 16. This figure illustrates a middle portion of a main channel comprising a 3D ion selective structure comprising charged beads, and electrode, and a bead bed structure comprising microslits. A voltage is applied across the 3D ion selective structure driving the formation of an IDZ concentrating the charged particles at the IDZ boundary. The side channel is positioned where the charged particles concentrate and divert said charged species away from the main channel, reducing the amount of said charged species from the fluid flowing through the main channel.

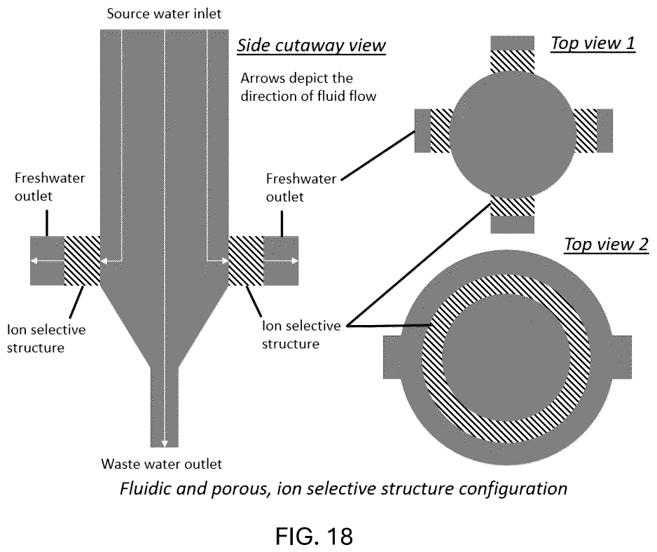

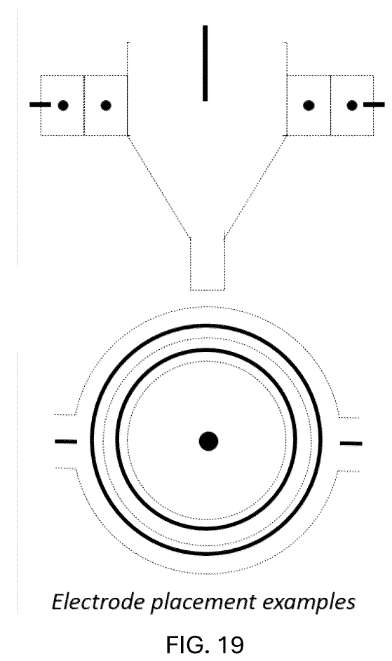

In an embodiment, the side channel is “on axis” and the main channel is diverted. With this embodiment, the main channel and ion selective structure can encircle or enclose the side channel. An exemplary device is shown in FIG. 18. A side view of this device is shown on the left with top views on the right. Exemplary electrode placement for the device in FIG. 18 is shown in FIG. 19.

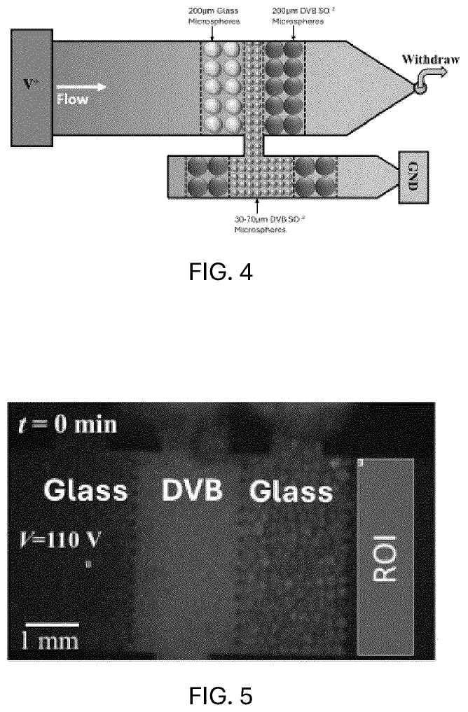

An exemplary device comprising a secondary channel is illustrated in FIG. 3 and FIG. 4. In FIG. 4, the fluidic device is configured so that a liquid flows through a main channel from the inlet, wherein a voltage is applied (signified with “V+”) and withdrawn from the outlet. The secondary channel is in fluid connection with the main channel. In this embodiment, the flow-through 3D ion selective structure comprises two different sizes of sulfonated DVB microbeads as defined by a bead bed structure. In this embodiment, the main channel further comprises a secondary bead bed structure comprising glass microbeads as defined by a bead bed structure. In this embodiment, the 3D ion selective structure and the secondary bead bed share a common portion of a bead bed structure. In this embodiment, a portion of the 3D ion selective structure, the 30-70 μm sulfonated DVB microbeads extend into the secondary channel, and the secondary channel further comprises sulfonated DVB microbeads. The secondary channel is grounded. In this exemplary device, a voltage is applied at the inlet while the secondary channel is grounded. IDZ and IEZ develop as shown in FIG. 3. In this out-of-plane voltage application, charged particles concentrate at the IEZ within the secondary channel and are removed from the fluid flowing towards the outlet, reducing the amount of charged particles from the liquid flowing through the main channel.

Another exemplary device comprising a secondary channel is illustrated in FIG. 2B. This figure is similar to the exemplary device depicted in FIG. 3 with the addition of a side channel. In FIG. 2B, the fluidic device is configured so that a liquid flows through a main channel from the inlet, wherein a voltage is applied and withdrawn from the outlet. The secondary channel is in fluid connection with the main channel. In this embodiment, the flow-through 3D ion selective structure extends into the secondary channel. The secondary channel is grounded. In this exemplary device, a voltage is applied at the inlet while the secondary channel is grounded. IDZ and IEZ develop as shown in FIG. 2B. In this out-of-plane voltage application, charged particles concentrate at the IEZ within the secondary channel and are removed from the fluid flowing towards the outlet, reducing the amount of charged particles from the liquid flowing through the main channel. Additionally, charged particles outside the IDZ in the main channel flow out through the side channel (or “waste” channel).

In an embodiment, the secondary channel comprises a side channel configured to divert concentrated species at the IEZ from the secondary channel.

As used herein, a main channel is referred to as a passageway configured for a liquid to flow from an inlet to an outlet. The width of a channel is referred to as the horizontal distance of the two points that are on the opposite edges of the cross-section perpendicular to the intended fluidic flow and are furthest away from each other. As used herein, the length of a channel is referred to as the distance from the inlet to the outlet through the channel along the intended fluid flow. As used herein, the height of the channel is referred to as the vertical distance from the floor of the channel to the ceiling of the same. When a main channel has a cylindrical shape, the diameter is referred to as the horizontal distance of the two points that are on the opposite edges of the cross-section perpendicular to the intended fluidic flow and are furthest away from each other.

In an embodiment, a main channel is referred to as having a width of greater than about 5 mm, a length of greater than about 10 mm, and a height greater than about 5 mm. In an embodiments, the main channel has a width greater than about 20 mm, greater than about 100 mm, or greater than about 1000 mm. In an embodiment, the main channel has a width of from about 5 mm to about 1000 mm, or from about 10 mm to about 500 mm. In an embodiment, the main channel is cylindrical and has a diameter of greater than about 5 mm, greater than about 20 mm, greater than about 100 mm, greater than about 1000 mm, from about 5 mm to about 1000 mm, or from about 10 mm to about 500 mm. The cross section of a main channel may have any two-dimensional shape, such as square, rectangular, circle, or a combination thereof. A main channel may be straight or curved. In an embodiment, the channel comprises pillars for support. The pillars extend from the floor of the channel to the ceiling of the main channel. The pillars may be of any shape, such as a round pillar. The size of the pillar is selected as to support the channel while not inhibiting the flow-through property of the device.

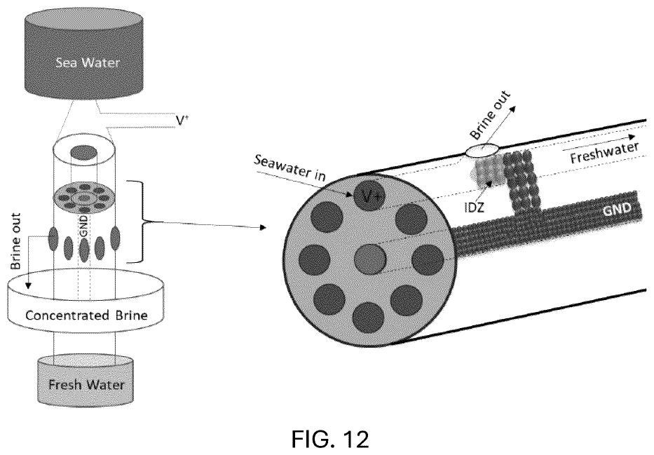

In an embodiment, the fluidic device comprises more than one main channel wherein each main channel is in fluid connection with a singular inlet and outlet. In an embodiment, at least two of the main channels comprise 3D ion selective structures in electrical connection. An exemplary fluidic device is shown in FIG. 12. In this figure, 8 main channels are in fluid connection with one inlet and one outlet. Each main channel comprises a 3D ion selective structure comprising microbeads. The microbeads are grounded from the same electrical connection (i.e. the microbeads in each of the main channel's 3D ion selective structure have the same electrical connection). The exemplary device illustrated in FIG. 12 is therein being utilized for water purification. Seawater flows from the inlet through the 3D ion selective structures of several main channels and toward the outlet. ICP focuses the charged particles at the boundary of the IDZ and into the side channel and purified water flows through the 3D ion selective structure and toward the outlet. A concentrated brine flows out of the branched channel (“brine out”) and desalted water flows to the outlet. FIG. 12 illustrates an exemplary embodiment of a device that comprises many parallel channels to reduce the impact of vortex flow. FIG. 12 shows a device wherein 8 parallel channels surround one grounded channel. Each parallel channel comprises a branched channel for the concentrated brine to flow out of the channel wherein freshwater continues toward the outlet. In this embodiment, voltage is applied near the inlet and each main channel comprises a secondary bead bed wherein the IDZ is formed. Each main channel comprises a side channel adjacent to the IDZ boundary wherein the concentrated charged species are diverted from the main channel.

The beads within the 3D ion selective structure or the secondary bead bed can be conductive or nonconductive. In an embodiment, the 3D ion selective structure comprises sulfonated divinylbenzene (DVB) beads. In an embodiment, the microbeads comprise ceramic materials. In an embodiment, the 3D ion selective structure or the secondary bead bed comprises glass beads. In an embodiment, the microbeads comprise chitosan. Beneficially chitosan adsorbs certain neutral species. In an embodiment, the microbeads comprise chitosan, polystyrene carboxylate, and/or silica carboxylate. Beneficially, all three have functional groups that can be modified to alter the surface charge and selectivity.

In an embodiment, the 3D ion selective structure is comprised of ceramics. In this embodiment, the 3D ion selective structure comprises disks, microbeads, and/or an inverse opal that comprise a ceramic material. In an embodiment, the 3D ion selective structure comprises disks, microbeads, and/or an inverse opal that comprise a ceramic material with a surface modification that yields a strongly negative zeta potential.

In an embodiment wherein the fluid comprises low-to moderate-salinity (not seawater), the 3D ion selective structure comprises glass porous disks and/or silica sand and/or a material of moderate thermal conductivity. Beneficially, these materials possess surface chemistry amenable to facile modification with anionic moieties.

In an embodiment, the 3D ion selective structure comprises a fiber network. Beneficially, fiber networks can be utilized as a sorbent and an ion exchange media and is adaptable for ICP-based electrokinetic separation.

The beads can be porous or nonporous. The pores can be nanoscale to microscale. In an embodiment, the microbeads are porous. In an embodiment, the microbeads are porous and the pores have a size ranging from about 10 nm to about 100 μm, or from about 10 nm to about 10 μm.

In an embodiment the 3D ion selective structure or the secondary bead bed comprise polymer coated microbeads. Beneficially, modification of the bead surfaces with polyelectrolytes like polystyrene sulfonate (anionic) and polyallylamine (cationic) can alter the surface charge and bead selectivity, thus influencing the IDZ formation.

In an embodiment, the secondary bead bed comprises beads to stabilize fluid flow and/or prevent the growth of large vortices. In an embodiment, the secondary bead bed comprises glass beads. In an embodiment, the microbead material within the secondary bead bed is selected such that the microbead itself does not carry much charge and is therefore more sensitive to certain charged species, furthermore, the charge of the microbeads may be modified to be positive or neutral depending on the desired sensitivity to a specific charged species.

In an embodiment, the microbeads in the 3D ion selective structure have a diameter of from about 1 μm to about 500 μm, or greater. In an embodiment, the microbeads in the secondary bead bed have a diameter of from about 1 μm to about 500 μm, or greater.

In some embodiments, the size of the beads and/or the length of the bed is determined by joule heating. Joule heating leads to the formation of large gas bubbles which interferes with the electric field and fluid flow affecting the device's stability and efficiency. Joule heating is directly related to the square of the total current adjusting the bead bed resistance addresses this concern. In some embodiments, joule heating can be mitigated by selecting smaller beads and/or increasing the bed length and/or using thermally conductive materials to enhance heat dissipation. In an embodiment, the device comprises a temperature monitoring system. Such a system tracks the internal temperature of the device. The temperature monitoring system can comprise any temperature measurement device as known in the art, for example one or more thermocouples or temperature-indicating labels.

In an embodiment, the system further comprises a sorbent material downstream of the 3D ion selective structure. In an embodiment, the system further comprises activated carbon downstream of the 3D ion selective structure to remove uncharged contaminants. In an embodiment, the system further comprises a filter downstream or upstream of the 3D ion selective structure to remove uncharged contaminants.

In some embodiments, the fluidic device further comprises a power source that is configured to have electrical communication with the electrodes at the inlet and/or outlet, and/or have electrical communication with the 3D ion selective structure. The power source is configured to supply DC with a voltage range from about 1 V to about 1000 V and any value in between. In an embodiment, the power supply is a battery.

In an embodiment, the inlet and/or outlet and/or 3D ion selective structure are in electrical contact with an electrode. Any electrode known in the art is acceptable, for example the electrode may comprise a planar electrode such as a thin metal film, pins and/or rods, wires, a wire mesh, a comb-like interdigitated or layered structure, and the like.

In some embodiments, the driving electrode is connected to the main channel by a glass frit. Beneficially, positioning a driving electrode in an adjunct port, connected to the main channel by a glass frit can dissipate heat, reduce Joule heating, and limit the formation of electrochemical side products.

Any method known in the art sufficient to transport the liquid through the main channel from inlet to outlet is acceptable for this device. In an embodiment, the inlet and outlet are configured such that there is uniform flow of the liquid through the main channel. As known in the art, there are various ways to ensure uniform flow, any one of which is acceptable. In an embodiment in the inlet and outlet are open reservoirs wherein uniform flow is gravity-driven, for instance by a fluid height differential, or a larger volume of fluid in the inlet than outlet, or by tilting the device such that the inlet is located in a higher plane than the outlet. In another embodiment, the inlet and outlet comprise tubing for fluid distribution. In an embodiment, fluid flow is ensured by a pump, or other device like a syringe. In an embodiment, the inlet serves as a port for a larger receptacle to plug into the inlet. In another embodiment, the outlet comprises a cotton plug, paper, a field of pillars, or other material or media to drive capillary flow through the device. In another embodiment, the inlet further comprises a filter to remove particulate matter such as debris or biological cells. In another embodiment the outlet comprises a receptacle to accept the liquid.

The inlet, outlet, and channels of the fluidic devices described herein can be comprised of any material as known in the art to house and transport a fluid. In some embodiments, the walls, floor, and/or ceiling of the main channel, side channel or secondary channel comprise a polymeric material. In some embodiments, the walls, floor, and/or ceiling of the fluidic channels comprise polydimethylsiloxane (“PDMS”), polymethylmethacrylate (“PMMA”), polystyrene, polycarbonate, cyclic olefin polymer, cyclic olefin copolymer, pressure sensitive adhesive tape, silicon, glass or the like. In an embodiment, the walls, floor, and/or ceiling of any of the channels comprise the resin of a 3D printer. In an embodiment, the walls, floor, and/or ceiling of any of the channels comprise polyethylene glycol. In another embodiment, the walls of any of the channels comprise crosslinked polyethylene glycol diacrylate (“PEGDA”) resin.

In an embodiment, the outlet comprises a storage reservoir to house the liquid. In an embodiment, the outlet comprises flowing the fluid to a point of use.

### Methods of Use

Methods described herein are meant to include any and all aspects and embodiments of the device and invention as described herein.

Disclosed herein are methods of selective transportation of ions through the use of ICP. Continuous separation of charged species from a flowing stream is achieved by diverting excluded species into a fluidic side channel or secondary channel. Disclosed herein are also methods of separation of neutral species from a flowing stream.

An exemplary method utilizing ICP to purify water with any of the devices described herein is shown in FIG. 3. In FIG. 3 an electrical bias is applied across a cation selective medium creating an IDZ in the main channel and an IEZ in a secondary channel. Purified water wherein the charged species have been reduced or removed continues to flow down the main channel. ICP captures or redirects charged particles at the boundary between the water and the IDZ, removing charged particles such as salts, bacteria, and oil-in-water emulsions with negative zeta potential.

The method comprises flowing a liquid through any of the devices as described herein. The liquid enters a main channel through an inlet, flows through the main channel, wherein the main channel comprises a 3D ion selective structure, to an outlet. A voltage is applied to inlet and/or outlet electrodes and/or the 3D ion selective structure. In an embodiment, an IDZ generated via ICP focuses charged particles at a boundary of an IDZ or in an IEZ.

In an embodiment, the method comprises flowing a liquid comprising a charged and/or neutral species through the main channel of the fluidic device as described herein and applying a voltage to the electrodes at the inlet and/or outlet and/or the 3D ion selective structure for a period of time focusing the charged species along an electric field gradient at the boundary of an ion depleted zone and/or in an ion enriched zone created by the 3D ion selective structure and removing at least a portion of the focused charged species from the liquid. In an embodiment, the charged species is removed from the liquid and into the side channel and/or the secondary channel.

The liquid can be any fluid comprising charged and/or neutral particles. In an embodiment, the liquid comprises water. In an embodiment, the liquid comprises seawater. In an embodiment, the liquid comprises blood. In an embodiment, the liquid comprises blood plasma. In an embodiment, the liquid comprises dialysate. In an embodiment, the liquid comprises a surfactant. In an embodiment, the surfactant comprises sodium dodecyl sulfate (SDS).

As used herein, a charged species or a charged particle is any particle, element, or compound with an electric charge. In an embodiment the charged particle comprises a salt, for example NaCl. In an embodiment, the charged species comprises serum albumin, creatinine, urea, glucose, cholesterol or a combination thereof.

In an embodiment, the method has a throughput of at least about 1 mL/hr, at least about 5 mL/hr, at least about 10 mL/hr, or at least about 100 mL/hr. In an embodiment, the method has a throughput of from about 1 mL/hr to about 3000 mL/hr, from about 10 mL/hr to about 3000 mL/hr, or from about 100 mL/hr to about 3000 mL/hr.

In an embodiment, the method comprises applying a voltage of from about 1V to about 1000V to the inlet and the 3D ion selective structure is grounded.

In an embodiment, the method comprises applying a voltage to the electrodes at the inlet and/or outlet and/or the 3D ion selective structure for at least about 1 min, at least about 5 minutes, at least about 8 minutes, at least about 10 minutes, or at least about 30 minutes. In an embodiment, the voltage is applied continuously for hours, days, or more. In an embodiment, the voltage is applied continuously for months, or more

In an embodiment, the method removes about 80% of the charged species from the liquid. In an embodiment, the method removes about 90% of the charged species from the liquid. In an embodiment, the method removes about 100% of the charged species from the liquid. In an embodiment, the method removes about 80% of the charged species from the liquid after about 1 minute, after about 2 minutes, after about 5 minutes, after about 8 minutes, or after about 10 minutes. In an embodiment, the method removes about 90% of the charged species from the liquid after about 1 minute, after about 2 minutes, after about 5 minutes, after about 8 minutes, or after about 10 minutes. In an embodiment, the method removes about 100% of the charged species from the liquid after about 1 minute, after about 2 minutes, after about 5 minutes, after about 8 minutes, or after about 10 minutes. In an embodiment, the method removes at least about 80% of the charged species from the liquid, at least about 85% of the charged species from the liquid, at least about 90% of the charged species from the liquid, at least about 95% of the charged species from the liquid, or at least about 100% of the charged species from the liquid.

In an embodiment, the method comprises applying equal voltages to the inlet and outlet electrodes, from about 1 V to about 1000 V, with the 3D ion selective structure grounded. In an embodiment, the electrode at the outlet is set at 0 V with the electrode at the inlet anywhere from about 250 mV to about 1000 V.

Disclosed herein are methods and systems for water purification comprising any of the devices or methods as disclosed herein.

Disclosed herein are methods of reducing charged species from spent dialysate comprising any of the devices or methods as disclosed herein.

In an embodiment, the method further comprises in-situ optical or non-optical detection and/or quantification of a focused or charged species.

In an embodiment, detection and/or quantification of the charged species is through optical means. In an embodiment, measurements are obtained using a fluorescence microscope and intensities quantified by comparison of IDZ to a subtracted background. In an embodiment, optical detection and/or quantification is through a colorimetric indicator, wherein detection and/or quantification are visual, for example by the naked eye or a microscope or a camera or a spectrometer or the like. In another embodiment, detection and/or quantification is through infrared absorption spectroscopy, ultraviolet absorption spectroscopy, radiometric imaging, Raman spectroscopy, interferometry, and the like, or combinations thereof.

In an embodiment, non-optical sensing is obtained through a change in measured impedance. In an embodiment, impedance is measured using an AC frequency sweep and a Nyquist plot or a Bode plot and the like. In an embodiment, change in impedance is detected by a detectable shift in current-voltage curves (“CVC”). Resistance dictated by ion transport to the electrode or by charge transfer reactions can be observed in the CVC. In an embodiment, non-optical sensing is obtained through conductivity measurements. In an embodiment, non-optical sensing is obtained through conductivity measurements at the inlet and/or outlet.

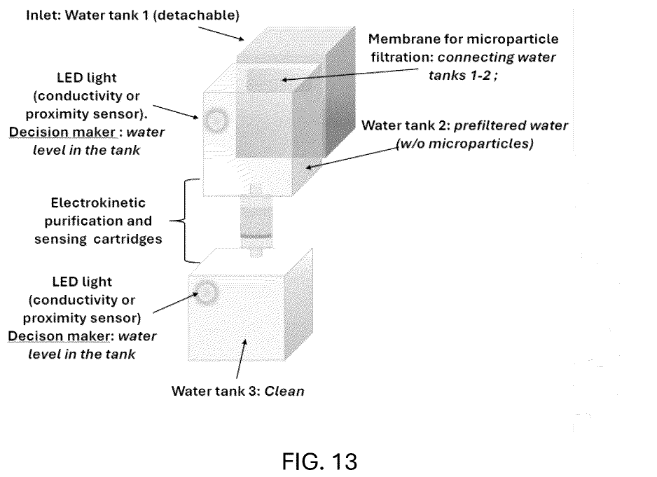

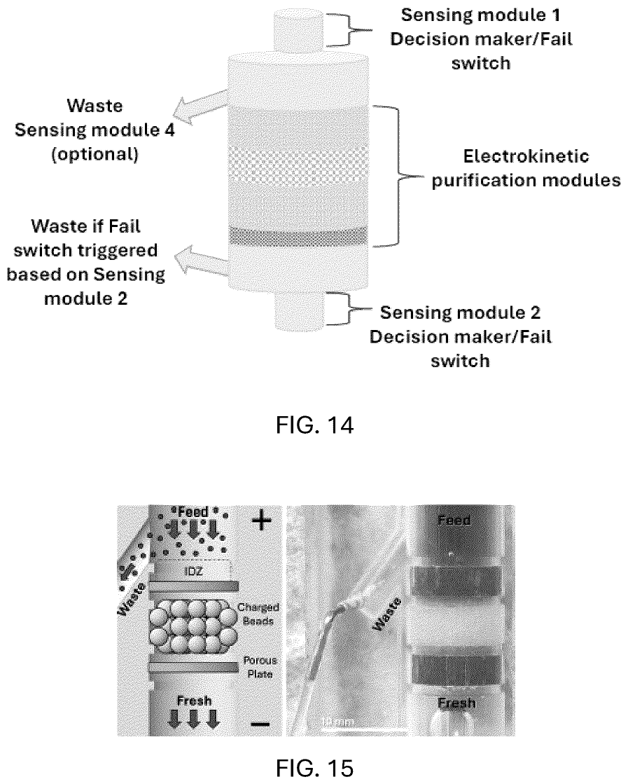

Disclosed herein are water purification systems and methods utilizing any of the devices and methods as disclosed herein and optionally any of the optical or non-optical detection and/or quantification of a focused or charged species as disclosed herein. Exemplary water purification systems are illustrated in FIG. 13 and FIG. 14. FIG. 13 and FIG. 14 illustrate systems wherein water flows through a device as described herein (electrokinetic purification modules) and further comprises filtration, and/or modules for sensing or detecting and/or quantifying water level, and/or modules for sensing or detecting any other quality of the water therein. In an embodiment, the non-optical sensing comprises sensing the surface charge of the 3D ion selective structure. In embodiments, the current may change as the beads or other parts of the 3D ion selective structure become fouled or damaged. In an embodiment, the sensing comprises measuring the current across the 3D ion selective structure.

Disclosed herein are kidney dialysis systems and methods utilizing any of the devices and methods as disclosed herein and optionally any of the optical or non-optical detection and/or quantification of a focused or charged species as disclosed herein.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1—Water Purification by Out-of-Line Voltage Application

A water-purification device was fabricated from Clear Microfluidics Resin (V7.0a) via a ProFluidics 285D DLP 3D printer and attached to a PDMS slab with tailored inlet and outlet reservoirs. Arrays of pillars spaced 100 μm apart with positioned in the fluidic channel to confine glass and sulfonated divinylbenzene (DVB) microbeads. The design of this device is illustrated in FIG. 4. Ion-selective transport governing beads, 30-70 μm in size are shown as light-gray beads and were sandwiched between 200 μm stabilizing glass beads shown as green. The device features an open reservoir for high and low-voltage electrodes to ensure efficient bubble removal upon contact with the atmosphere. High voltage application produces gas bubbles due to hydrolysis, and Joule heating enlarges these bubbles, disrupting the electrical connection and occasionally blocking the central channel. Open reservoirs allow electrodes with a larger surface area and ensure bubbles burst upon contact with the atmosphere. These reservoirs are interconnected with the central fluidic channel via each 500 μm wide channel.

As shown in FIG. 4, an electric field was applied out-of-line of the pressure driven flow. A saline solution flowed through the device and a voltage of 10V was applied for 10 minutes. The out-of-line voltage application design maintains the IDZ for an extended duration and exhibits improved desalination.

The driving voltage was applied across the bead bed using a Programmable DC Power Supply (Keithley 2260B-250-4, Keithley Instruments) connected to Pt electrode wires in the reservoirs. To facilitate and regulate a flow of 5-50 μL/min, two syringe pumps were employed.

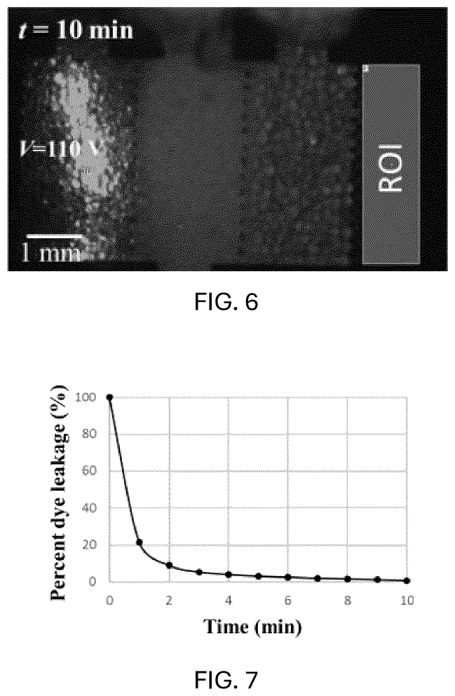

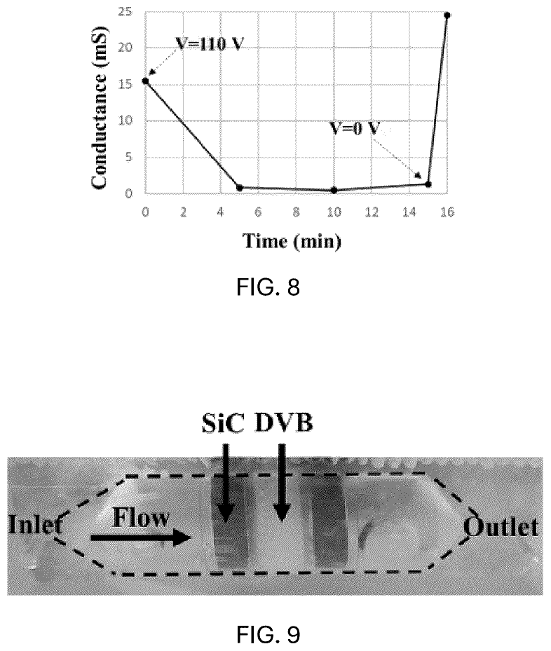

FIGS. 5 and 6 show the fluorescence distribution before and after the voltage was applied to induce ICP in the fluidic device, demonstrating significant desalination. FIG. 5 is a micrograph showing the fluorescence distribution of the central channel before the application of voltage. FIG. 6 is a micrograph showing the fluorescence distribution of the central channel 10 minutes after 10V was applied. This is confirmed by the change in dye leakage percentage shown in FIG. 7. A rectangular region of interest (ROI) was drawn to assess the percentage of dye leakage downstream to evaluate the desalting performance. 92% salt removal was achieved after application of the voltage for 10 minutes, validated by conductivity measurements. FIG. 8 is a graph of the conductance of the downstream filtered water. The conductance was restored to its previous value once the electric field was removed.

The out-of-line voltage application resulted in 32-fold reduction in the conductance of the desalted stream within 10 minutes.

### Example 2—Scaling Water Purification

To scale up water purification, a fully 3D-printed device was designed by bonding two 3D thermoplastic pieces with epoxy adhesive, housing two porous SiC ceramic disks with 15 μm nominal pore size in a 10 mm diameter channel. This design is shown in FIG. 9. SiC's high thermal conductivity helped to dissipate thermal energy generated from joule heating. Additionally, the SiC disks act as a confinement barrier for sulfonated DVB beads. The DVB beads were 30-70 μm in size and serve as the primary ion-selective feature.

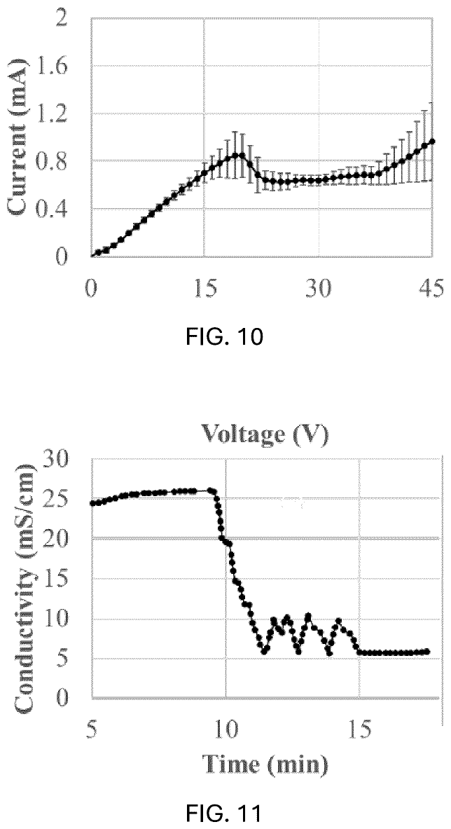

100 mM NaCl feed solution was infused into the inlet with withdrawn from the outlet, and 100V applied across the device. A current-voltage plot is shown in FIG. 10. The plot in FIG. 10 exhibits distinct limiting and over-limiting regimes, characteristic of ICP. 80% salt removal from a 100 mM NaCl feed solution was demonstrated with 100V application within about 8 minutes, as shown in FIG. 11.

### Example 3—Gravity Driven Water Purification

A fluidic device as illustrated in FIG. 15 was used for this Example. FIG. 15 shows an illustration of the fluidic device on the left and a photograph of the fluidic device on the right. Charged beads are sandwiched between porous ceramic plates to dissipate heat and stabilize fluid flow at the separation interface. Volage was applied across the bead bed to create an IDZ as shown in FIG. 15. A concentrated waste stream flowed out of the device through a branched channel and purified water flowed through the device and to the outlet.

### Example 4—Separation Characteristics of Blood Plasma

A fluidic device as illustrated in FIG. 16 was used for this Example. As shown in FIG. 16, the main channel comprises a branched channel upstream of the bead bed. The ion selective beads were made of chitosan for IDZ formation and neutral waste adsorption. Micro slits on either side of the bead bed trap and hold the beads in place and stabilize the IDZ boundary, in this example at the junction of the branch. Electrodes are located under and adjacent to the bead bed to apply the voltage required to drive ICP.

The separation characteristics of charged and neutral compounds in blood plasma was analyzed by using fluorescent tracers. Texas red dye-linked albumin to represent charged species and BODIPY FL to represent neutral species.

FIG. 17 contains fluorescence micrographs showing the location of tracers under 40V. The micrographs on the left, labeled a and b, are for undiluted blood plasma samples and the micrographs on the right, labeled c and d, are for model blood plasma containing 0.1% of the albumin content of blood plasma. The dye-linked albumin (red, top row) is redirected into the branched channel in both cases. BODIPY FL was redirected in undiluted plasma only. Neutral BODIPY FL fluorescent dye is lipophilic. Without being limited to a particular theory or mechanism, it is contemplated that BODIPY FL is interacting with lipids and other charged lipophilic compounds present in human blood plasma and is thereby being directed into the upper branch in the undiluted plasma. Neutral compounds native to blood may be carried into the charged stream by way of intermolecular interactions between these compounds and charged macromolecules present in blood plasma.

### Example 6—Neutral Species Present in Blood

Neutral species depletion will be characterized in various systems. The retention of neutral BODIPY FL dye and mesityl oxide (a neutral tracer used for capillary electrophoresis to represent neutral species) during ICP in sample solutions (carbonate or phosphate buffer) with varying concentrations of protein (albumin), lipids (fatty acids), and polyelectrolyte (polystyrene sulfonate) will be assessed. The influence of surfactants (sodium dodecyl sulphate, SDS) on neutral species depletion mechanism will be investigated. In micellar electrokinetic capillary chromatography (MEKC), formed micelles act as pseudo-stationary phase and interact with analytes based on their hydrophobicity. In such a case, compounds that are not ionized under experimental conditions can be incorporated into the micelle and electrophoretically separated due to the charge of the micelles.

### Example 7—Separation Characteristics of Spent Dialysate

Spent dialysate samples spiked with fluorescent tracers will be employed to characterize the separation. Additionally, the composition of spent dialysate before and after ICP will be characterized by employing LCMS and chemical assays focusing on solutes that are most abundant and characteristic for kidney disease patients (e.g. serum albumin, creatinine, urea, glucose, cholesterol).

### Example 8—Scaled Up Device

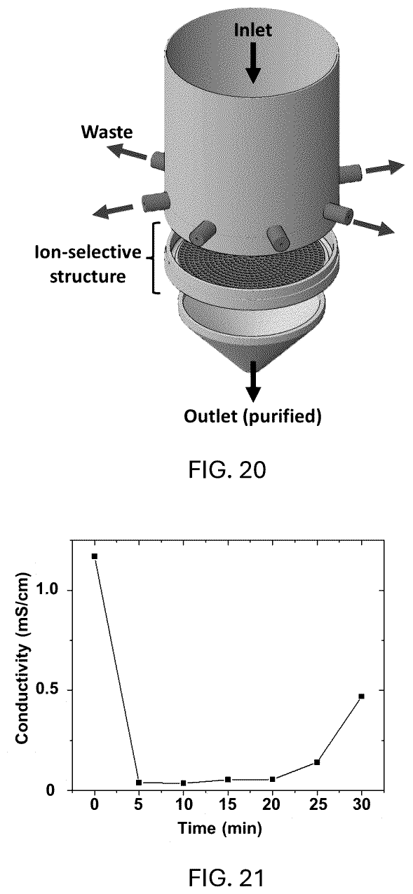

A larger device according to FIG. 20 was utilized in this Example. This device had a main channel with a diameter of 10 cm and a length of 15 cm. The side channels had a diameter of 1.5 cm. The ion selective structure comprised two 100 mm porous SiC discs (100 diameter, 40% porosity, 30 μm pore size, 120 W/mK thermal conductivity) to confine 30-70 μm sulfonated-DVB beads. Two platinum-coated titanium mesh electrodes were placed across the pack beads to apply an electrical bias and induce ICP. Cylindrical conduits were placed along the periphery of the upstream ceramic disk to channel the brine stream outside the device. A cone-shaped conduit facilitated the collection of fresh water downstream for analysis.

The fluid was an electrolyte solution with 10 nM NaCl. The device utilized gravity flow conditions. The estimated gravity-driven flow rate was 5 mL/min. Pt/Ti mesh (50×50 mm) driving electrodes were positioned on both sides of the packed bed and SiC discs.

Conductivity change over time is shown in FIG. 21. Output water aliquots were collected at 5-minute time points, and conductivity was measured. EDAQ, isoPod was used for continuous monitoring of solution conductivity with a two-electrode conductivity probe. Current voltage curves (CVCs) were obtained using a Keithley multimeter. DC voltage sweep was applied (0 -120 V, 0.1 V/s) between the channel inlet and outlet, using a power supply (2450 SourceMeter®, Keithley Instruments, Cleveland, OH). Simultaneously, the I-V curve was recorded using Kick Start software (Keithley Instruments, Cleveland, OH).

A maximum of 97 % water desalination was observed after 10 min of driving voltage application (100V).

### Example 9—Single Bead Bed with Al₂O₃ Discs

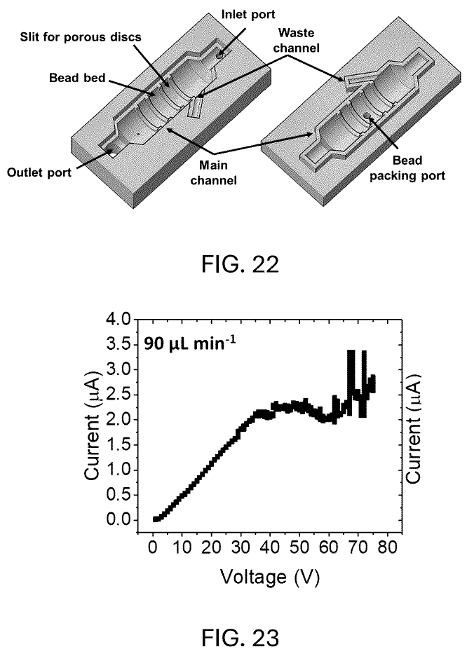

This Example utilized a device according to FIG. 22. The main channel was 30 mm long and 10 mm wide. The ion-selective structure consisted of two porous Al₂O₃ discs and a bead bed of sulfonated DVB polystyrene beads.

A solution of 10 mM NaCl was directed through the device with 30 and 90 μL/min withdraw flow conditions. Cu wire and stainless-steel electrodes were positioned in the input and output ports, respectively. The driving voltage was varied, either 30 V, 45 V or 60 V.

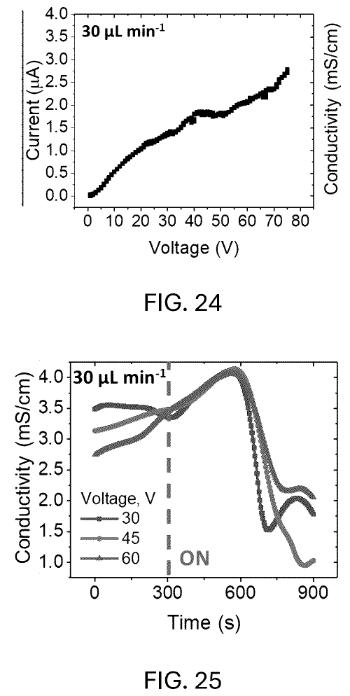

A CVC curve obtained using 90 μL/min withdraw flow rate is shown in FIG. 23 and a CVC curve obtained using 30 μL/min withdraw flow rate is shown in FIG. 24. The conductivity decrease of the output water over time after the application of the driving voltage using 30 μL/min withdraw flow rate is shown in FIG. 25.

No significant transition between limiting and overlimiting behavior was observed in CVC curve obtained at a 30 μL/min flow rate. Transition between ohmic and limiting behavior was observed at 30V. At a 90 μL/min flow rate, limiting and overlimiting behavior were observed at 30 and 60V, respectively. After application of the driving voltage, a moderate decrease in output water conductivity was observed. Maximum purification efficiency was 53% at 90 μL/min, 60V, and 77% at 30 uL/min, 45V conditions.

### Example 10—Single Bead Bed with SiC Discs

This Example utilized a device according to FIG. 22. The ion-selective structure consisted of two porous discs and a single bead bed. The ion-selective structure consisted of SiC discs and a bead bed of sulfonated DVB polystyrene beads.

A solution of 10 mM NaCl was directed through the device with 30 and 90 μL/min withdraw flow conditions. Cu wire and stainless-steel electrodes were positioned in the input and output ports, respectively. The driving voltage was varied, either 30 V, 45 V or 60 V.

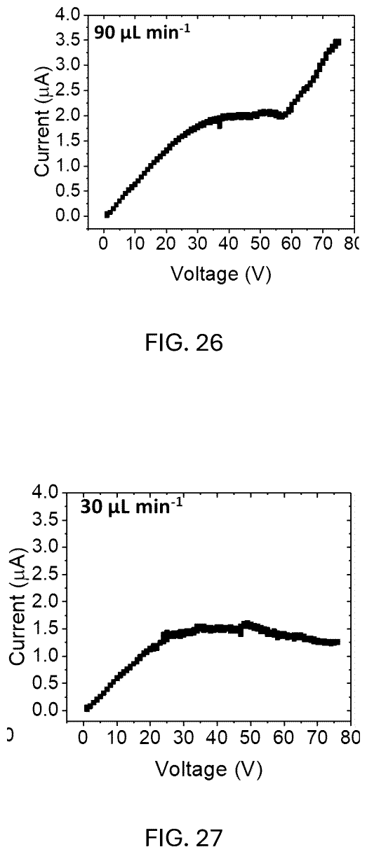

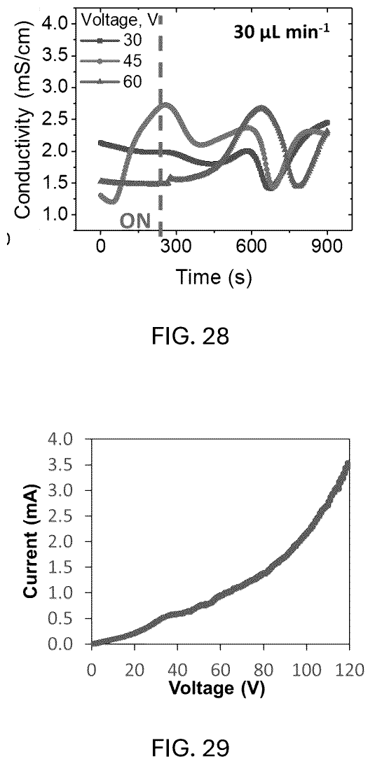

A CVC curved obtained using 90 μL/min withdraw flow rate is shown in FIG. 26 and a CVC curve obtained using 30 μL/min withdraw flow rate is shown in FIG. 27. The normalized water desalination efficiency is shown in FIG. 28.

Transition between ohmic and limiting behavior was observed at 30V at 90 μL/min and 20V at 30 μL/min flow rates. At a 90 μL/min flow rate, limiting and overlimiting behavior transition was observed at 60V. After application of the driving voltage, a moderate decrease in output water conductivity was observed.

Maximum purification efficiency observed was 69% at 90 μL/min and 45V, and 56% using 30 μL/min flow rate and 45V. The instability of the salt removal efficiency was due to bubble formation at the electrodes. To address this, frits and additional frit wells were incorporated in the device design to separate the electrode from the main fluid flow.

### Example 11—Addition of Frits

This Example utilized a device according to FIG. 22, with the addition of a port near the inlet wherein the driving electrode is connected to the main channel by a glass frit. Glass frits (⅛″) were obtained from BASi (West Lafayette, IN). Glass frits were secured into thermoplastic wells with housing ports using epoxy adhesive.

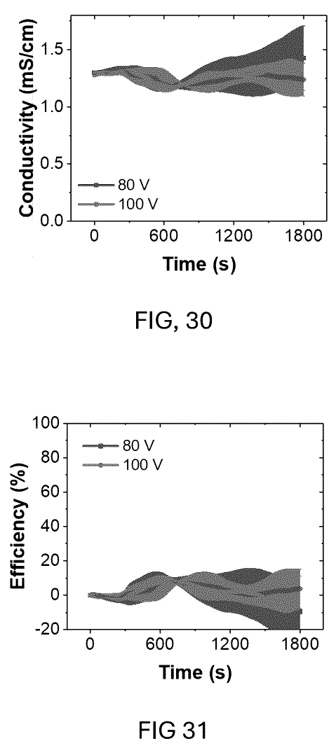

First, no discs or beads were utilized. A solution of 10 mM NaCl was directed through the device with 30 μL/min withdraw flow conditions. Pt electrodes were positioned in frit wells. Applied voltage was either 80V or 100V. A CVC curve obtained using 30 μL/min withdraw flow rate is shown in FIG. 29, conductivity decrease over time after application of the driving voltage is shown in FIG. 30. Salt removal efficiency depicted as a percent increase over time is shown in FIG. 31. No significant transition between ohmic, limiting, and over-limiting behavior was observed in CVC curves. After application of the driving voltage, no significant decrease in output water conductivity was observed. Average desalination efficiency was around 5% over 30 minutes. Standalone glass frits do not generate strong ICP and IDZ formation; glass frits do not significantly contribute to the desalination of the output water stream.

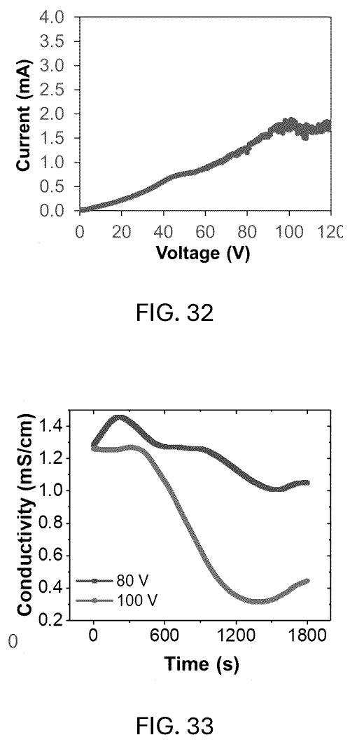

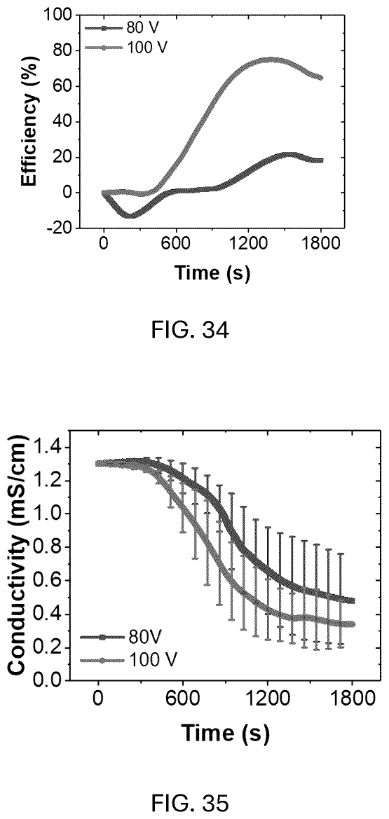

Next, a device with frits and SiC discs only, and no beads was tested. A solution of 10 mM NaCl was directed through the device with 30 μL/min withdraw flow conditions. Pt electrodes were positioned in frit wells. Applied voltage was either 80V or 100V. A CVC curve obtained using 30 μL/min withdraw flow rate is shown in FIG. 32. Conductivity decrease over time after application of the driving voltage is shown in FIG. 33. Salt removal efficiency depicted as a percent increase over time is shown in FIG. 34. No significant transition between limiting and overlimiting behavior was observed. However, significant conductivity decrease was observed. The maximum desalination efficiency reached was 80% and 20% after application at 100V and 80V over 30 30-minute time period, respectively. Standalone SiC discs can serve as ion-selective structures for ICP generation.

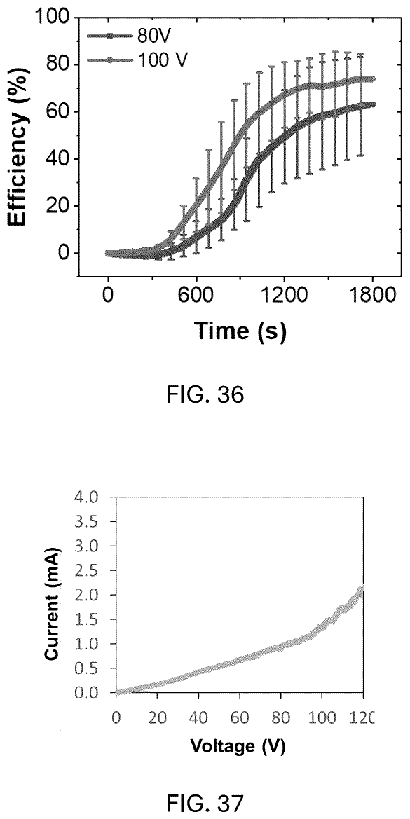

Next, a device with frits, SiC discs, and acid-washed SiO₂ beads (30-70 μm) was tested. A solution of 10 mM NaCl was directed through the device with 30 μL/min withdraw flow conditions. Pt electrodes were positioned in frit wells. Applied voltage was either 80V or 100V. Conductivity decrease over time after application of the driving voltage is shown in FIG. 35. Salt removal efficiency depicted as a percent increase over time is shown in FIG. 36. After application of the driving voltage, a significant decrease in output water conductivity was observed. 85-87% of maximum desalination efficiency was achieved within 30 minutes after application of 80 and 100 V driving voltage, with average performance of 75% and 62% across three separate trials.

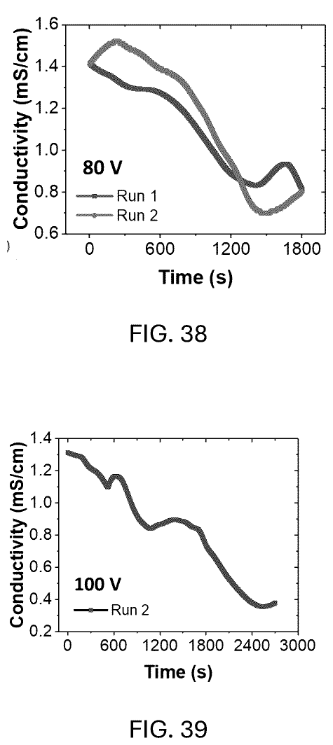

Next, a device with frits, SiC discs, and AlN beads (32 μm) was tested. A solution of 10 mM NaCl was directed through the device with 30 μL/min withdraw flow conditions. Pt electrodes were positioned in frit wells. Applied voltage was either 80 V or 100 V. A CVC curve is shown in FIG. 37. Conductivity decrease over time after application of the driving voltage is shown in FIG. 38 and FIG. 39. No significant transition between ohmic, limiting, and over-limiting behavior was observed in CVC curves. After application of the driving voltage (80 V), a significant decrease in output water conductivity was observed. Approximately 55% desalination efficiency was achieved when 80 V was applied. Further improvement of desalination was achieved by applying 100 V, wherein salt removal efficiency improved to 69%.

### Example 12—Lengthened Bead Bed

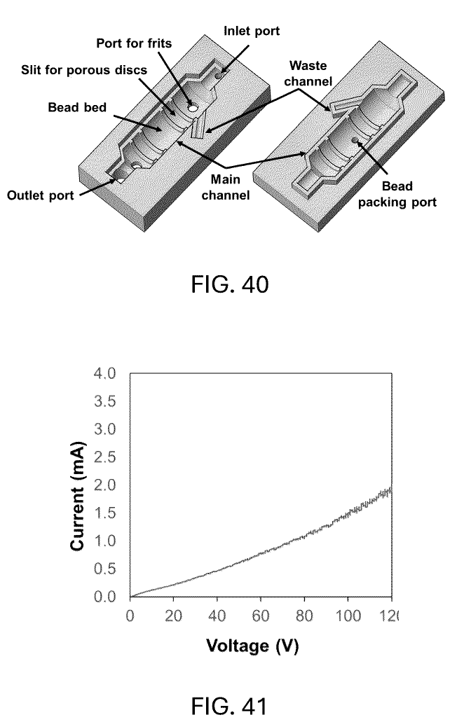

This Example utilized a device according to FIG. 40. The main channel had a length of 30 mm and a width of 10 mm. The ion-selective structure consisted of two porous discs and a bead bed with increased length.

This test used SiC discs and acid washed glass beads. This test also incorporated frits. A 10 mM NaCl was directed through the device with a 30 μL/min withdrawn flow rate. Applied voltage was 100 V.

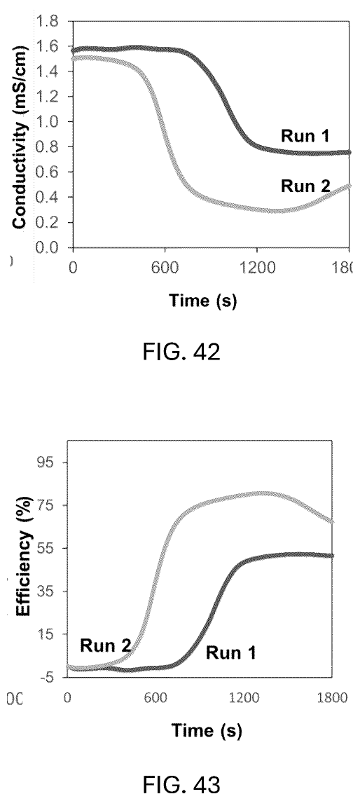

A CVC curve is shown in FIG. 41. Conductivity decrease over time after application of the driving voltage 100 V is shown in FIG. 42. Salt removal efficiency depicted as percent increase over time is shown in FIG. 43.

No significant transition between ohmic, limiting, and over-limiting behavior was observed in CVC curves. After application of the driving voltage, a significant decrease in output water conductivity was observed. Maximum desalination efficiency was 77%. On average, between two trials, ~60% desalination efficiency was achieved.

### Example 13—Two Bead Beds

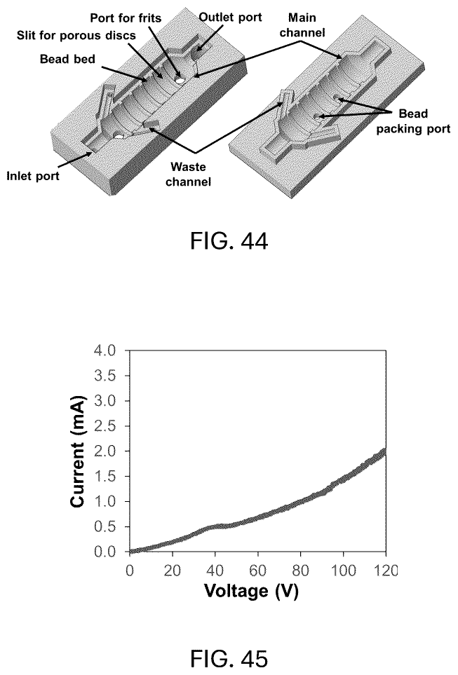

This Example utilized a device according to FIG. 44. The ion-selective structure consisted of three SiC discs and two separate bead beds. Beneficially, with this scheme, each bed can be packed with similar or different beads, increasing the tunability of the device based on the intended application. AlN beads (30 μm) and activated carbon beads (100 μm) were used in this Example.

A 10 mM NaCl was directed through the device with a 30 μL/min withdrawn flow rate. Applied voltage was 80 V or 100 V.

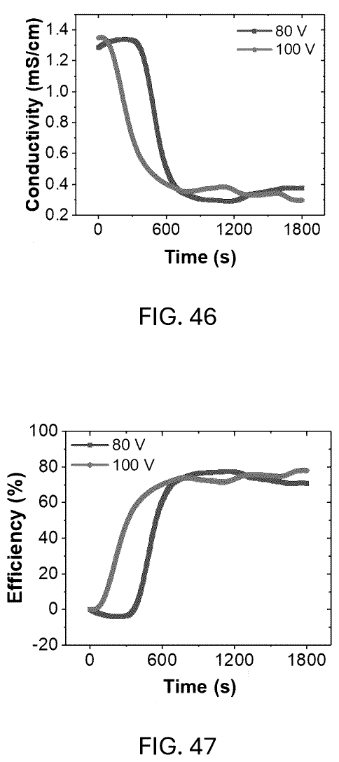

CVC curve is shown in FIG. 45. Conductivity decrease over time after application of the driving voltage 80 and 100V is shown in FIG. 46. Salt removal efficiency depicted as a percent increase over time is shown in FIG. 47. No significant transition between ohmic, limiting, and overlimiting behavior was observed in CVC curves. After application of the driving voltage, a significant decrease in output water conductivity was observed. Approximately 80% of the desalination efficiency was achieved.

### Example 14—High Salination

This Example utilized a device according to FIG. 44. The ion-selective structure consisted of three SiC discs and two separate bead beds with AlN beads (30 μm) and activated carbon beads (100 μm).

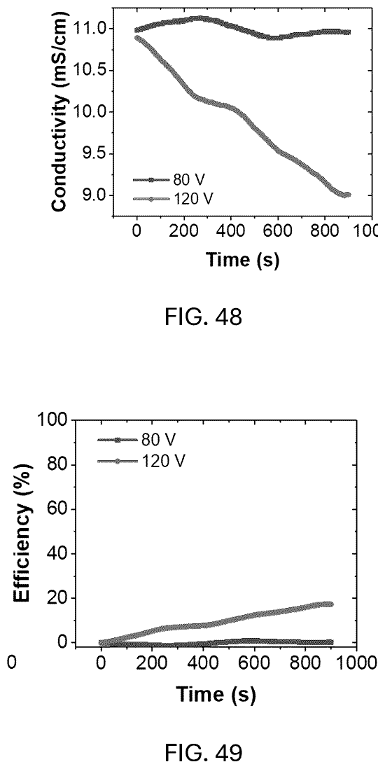

A 100 mM NaCl solution was directed through the device with a 30 μL/min withdraw flow rate. Applied voltage was 80 V or 120 V. Conductivity decrease over time is shown in FIG. 48 and salt removal efficiency depicted as a percent decrease over time is shown in FIG. 49.

No significant decrease in conductivity was observed when 80 V driving voltage was used. After application of 120 V, a decrease in output water conductivity was observed. Water desalination efficiency reached 20% within the 30-minute experiment. These results indicate that water desalination is feasible using higher salinity input water (for example, dialysate, seawater), but to reach matching purification efficiency, higher operating voltages, and therefore power consumption, may be required.

The present disclosure is further defined by the following numbered embodiments:
1. A fluidic device comprising a main channel connected to at least one inlet and at least one outlet, wherein the main channel is configured to flow a liquid through the main channel from inlet to outlet; at least one flow-through 3D ion selective structure within the main channel comprising microbeads, wherein the microbeads are contained within a bead bed which extends at least a portion of the width and length of the main channel as defined by a bead bed structure; a side channel configured to flow a portion of the liquid within the main channel from the main channel to a side outlet, wherein the side channel is located upstream from the 3D ion selective structure; wherein the inlet and/or the outlet is connected to an electrode.
2. A fluidic device comprising a main channel connected to at least one inlet and at least one outlet, wherein the main channel is configured flow a liquid through the main channel from inlet to outlet; a secondary channel adjacent to the main channel and in fluid connection with the main channel; at least one flow-through 3D ion selective structure comprising microbeads, wherein the microbeads are contained within a bead bed which extends at least a portion of the width and length of the main channel and at least a portion of the width and length of the secondary channel as defined by a bead bed structure; wherein the inlet and/or the outlet is connected to an electrode.
3. The fluidic device of embodiment 2, further comprising a side channel configured to flow a portion of the liquid within the main channel from the main channel to a side outlet, wherein the side channel is located upstream from the 3D ion selective structure.
4. The fluidic device of embodiment 2 or embodiment 3, further comprising a side channel configured to flow a portion of the liquid within the secondary channel from the secondary channel to a side outlet.
5. The fluidic device of any one of embodiments 1-4, wherein at least a portion of the 3D ion selective structure is in contact with an electrode, and wherein at least a portion of the electrode extends outside of the main channel and/or secondary channel for electrical connection.
6. The fluidic device of any one of embodiments 1-5, wherein the device has more than one main channel and wherein the main channels are connected to the same inlet and the same outlet, or several distinct inlets and several distinct outlets.
7. The fluidic device of embodiment 6, wherein each main channel has at least one flow-through 3D ion selective structure.
8. The fluidic device of embodiment 7, wherein each flow-through 3D ion selective structure has a separate electrical contact.
9. The fluidic device of embodiment 7, wherein one or more flow-through 3D ion selective structure has the same electrical contact.
10. The fluidic device of any one of embodiments 1-9, wherein the main channel further comprises at least one secondary bead bed comprising microbeads, wherein the microbeads are contained within the secondary bead bed defined by a bead bed structure within the main channel.
11. The fluidic device of embodiment 10, wherein the secondary bead bed is located upstream from the flow-through 3D ion selective structure.
12. The fluidic device of any one of embodiments 10-11, wherein the microbeads in the secondary bead bed are not conductive.
13. The fluidic device of any one of embodiments 1-12, wherein the bead bed structure comprises bead bed posts wherein the bead bed posts extend from the floor of the main channel and are spaced such that the microbeads are contained within the area defined by the bead bed posts.
14. The fluidic device of any one of embodiments 1-13, wherein the bead bed structure comprises a weir structure, a porous matrix or material, a chemical linker, or combination thereof.
15. The fluidic device of any one of embodiments 1-14, wherein the bead bed structure comprises porous ceramic plates.
16. The fluidic device of any one of embodiments 1-15, wherein the device further comprises a power source connected with the inlet electrode and/or the outlet electrode and/or the 3D ion selective structure, wherein the power source is configured to supply a voltage in the range of from about 1 V to about 1000 V.
17. The fluidic device of embodiment 16, wherein the power source is a battery.
18. The fluidic device of any one of embodiments 1-17, wherein the 3D ion selective structure comprises a planar microband electrode, a rod, a wire, a pin, or combinations thereof.
19. The fluidic device of any one of embodiments 1-18, wherein the 3D ion selective structure comprises a conductive epoxy, an ionic liquid, or combinations thereof.
20. The fluidic device of any one of embodiments 1-19, wherein the microbeads in the 3D ion selective structure have a diameter of from about 1 μm to about 500 mm.
21. The fluidic device of any one of embodiments 10-20, wherein the microbeads in the secondary bead bed have a diameter of from about 1 μm to about 500 mm.
22. The fluidic device of any one of embodiments 1-21, wherein the microbeads in the 3D ion selective structure comprise sulfonated divinylbenzene.
23. The fluidic device of any one of embodiments 1-22, wherein the microbeads in the 3D ion selective structure comprise chitosan.
24. The fluidic device of any one of embodiments 10-23, wherein the microbeads in the secondary bead bed comprise glass.
25. A method of removing charged species from a liquid comprising flowing a liquid containing at least one charged species through the main channel of the fluidic device of any one of embodiments 1-24; applying a voltage to the inlet and/or outlet and/or the 3D ion selective structure for a period of time focusing the charged species along an electric field gradient at the boundary of an ion depleted zone and/or in an ion enriched zone created by the 3D ion selective structure; and removing at least a portion of the focused charged species from the liquid, either into the side channel or into the secondary channel.
26. The method of embodiment 25, wherein the liquid is fresh water or seawater.
27. The method of embodiment 25, wherein the liquid is blood or blood plasma.
26. A method of purifying water comprising flowing water comprising at least one charged species through the main channel of the fluidic device of any one of embodiments 1-24; applying a voltage to the inlet and/or outlet and/or the 3D ion selective structure for a period of time focusing the charged species along an electric field gradient at the boundary of an ion depleted zone and/or in an ion enriched zone created by the 3D ion selective structure; and removing at least a portion of the focused charged species from the water, either into the side channel or into the secondary channel.
27. The method of embodiment 26, wherein the charged species is a salt.
28. The method of embodiment 26 or 27, wherein the water is seawater.
29. A method of removing charged species from spent dialysate comprising flowing blood or blood plasma comprising at least one charged species through the main channel of the fluidic device of any one of embodiments 1-24; applying a voltage to the inlet and/or outlet and/or the 3D ion selective structure for a period of time focusing the charged species along an electric field gradient at the boundary of an ion depleted zone and/or in an ion enriched zone created by the 3D ion selective structure; and removing at least a portion of the focused charged species from the spent dialysate.
30. The method of embodiment 29, wherein the charged species comprise serum albumin, creatinine, urea, glucose, cholesterol or a combination thereof.
31. The method of any one of embodiments 25-30, wherein the method has a throughput of at least about 1 mL/hr.
32. The method of any one of embodiments 25-31, wherein the method has a throughput of from about 1 mL/hr to about 3000 mL/hr.
33. The method of any one of embodiments 25-32, wherein from about 1V to about 1000V, or from about 10V to about 100V is applied to the inlet and the 3D ion selective structure is grounded.
34. The method of any one of embodiments 25-33, wherein the voltage is applied for about 1 minute to about 30 minutes, or more.
35. The method of any one of embodiments 25-33, wherein the voltage is applied continuously for days, or months, or more.
36. The method of any one of embodiments 25-35, wherein the method removes about 80% of the charged species from the liquid.
37. The method of any one of embodiments 25-36, wherein the method removes about 90% of the charged species from the liquid.
38. The method of any one of embodiments 25-37, wherein the method removes about 100% of the charged species from the liquid.
39. A water purification system comprising the fluidic device of any one of embodiments 1-24.
40. A kidney dialysis system comprising the fluidic device of any one of embodiments 1-24.

The above disclosure is intended to be illustrative and not exhaustive. This description will suggest many variations and alternatives to one of ordinary skill in this art. All these alternatives and variations are intended to be included within the scope of the following claims where the term “comprising” means “including, but not limited to”. Those familiar with the art may recognize other equivalents to the specific embodiments described herein which equivalents are also intended to be encompassed by the following claims.

What is claimed is:

1. A fluidic device comprising:
a main channel connected to at least one inlet and at least one outlet, wherein the main channel is configured to flow a liquid through the main channel from inlet to outlet, wherein the inlet and/or the outlet is connected to an electrode;
at least one flow-through 3D ion selective structure within the main channel comprising microbeads, wherein the microbeads are contained within a bead bed which extends at least a portion of the width and length of the main channel as defined by a bead bed structure; and
one or more of the following:
a) a side channel configured to flow a portion of the liquid within the main channel from the main channel to a side outlet, wherein the side channel is located upstream from the 3D ion selective structure; and
b) a secondary channel adjacent to the main channel and in fluid connection with the main channel, wherein at least a portion of the bead bed and bead bed structure extends into at least a portion of the secondary channel.

2. The fluidic device of claim 1, wherein at least a portion of the 3D ion selective structure is in contact with an electrode, and wherein at least a portion of the electrode extends outside of the main channel and/or secondary channel for electrical connection.

3. The fluidic device of claim 1, wherein the device has more than one main channel and wherein the main channels are connected to the same inlet and the same outlet, or several distinct inlets and several distinct outlets, wherein each main channel has at least one flow-through 3D ion selective structure.

4. The fluidic device of claim 1, wherein the main channel further comprises at least one secondary bead bed comprising microbeads, wherein the microbeads are contained within the secondary bead bed defined by a bead bed structure within the main channel, wherein the secondary bead bed is located upstream from the flow-through 3D ion selective structure.

5. The fluidic device of claim 4, wherein the microbeads in the secondary bead bed are not conductive and/or comprise glass.

6. The fluidic device of claim 1, wherein the bead bed structure comprises a weir structure, a porous matrix or material, a chemical linker, porous ceramic plates, bead bed posts wherein the bead bed posts extend from a surface of the main channel and are spaced such that the microbeads are contained within the area defined by the bead bed posts, or a combination thereof.

7. The fluidic device of claim 1, wherein the device further comprises a power source connected with the inlet electrode and/or the outlet electrode and/or the 3D ion selective structure, wherein the power source is configured to supply a voltage in the range of from about 1 V to about 1000 V.

8. The fluidic device of claim 1, wherein the 3D ion selective structure comprises a planar microband electrode, a rod, a wire, a pin, a conductive epoxy, an ionic liquid, or combinations thereof.

9. The fluidic device claim 1, wherein the microbeads in the 3D ion selective structure have a diameter of from about 1 μm to about 500 mm.

10. The fluidic device of claim 1, wherein the microbeads in the 3D ion selective structure comprise sulfonated divinylbenzene, chitosan, or a combination thereof.

11. A method of removing charged species from a liquid comprising:
flowing a liquid containing at least one charged species through the main channel of the fluidic device of claim 1;
applying a voltage to the inlet and/or outlet and/or the 3D ion selective structure for a period of time focusing the charged species along an electric field gradient at the boundary of an ion depleted zone and/or in an ion enriched zone created by the 3D ion selective structure; and
removing at least a portion of the focused charged species from the liquid, into the side channel and/or into the secondary channel.

12. The method of claim 11, wherein the liquid is fresh water, seawater, blood, blood plasma, or dialysate.

13. The method of claim 11, wherein the charged species is a salt and the liquid is water or seawater.

14. The method of claim 11, wherein the charged species comprise serum albumin, creatinine, urea, glucose, cholesterol or a combination thereof and the liquid is spent dialysate.

15. The method of claim 11, wherein the method has a throughput of from about 1 mL/hr to about 3000 mL/hr.

16. The method of claim 11, wherein from about about 10V to about 1000V is applied to the inlet and the 3D ion selective structure is grounded.

17. The method of claim 11, wherein the voltage is applied for at least about 30 minutes, for days, or months, or more.

18. The method of claim 11, wherein the liquid contains at least one neutral species and removing at least a portion of the neutral species from the liquid.

19. The method of claim 11, wherein the method removes at least about 80% of the charged species from the liquid.

20. A water purification system or a kidney dialysis system comprising the fluidic device of claim 1.
